(19) United States
(12) Patent Application Publication
(10) Pub. No.: US 2021/0093230 A1
(43) Pub. Date: Apr. 1, 2021

(54) DEVICE FOR DETERMINING A PARAMETER OF THE LOCOMOTION OF ANIMALS

(71) Applicant: 4DVets AG, Frenkendorf (CH)

(72) Inventors: Erik SCHKOMMODAU, Liestal (CH); Patrick BLAETTLER MONNIER, Waldenburg (CH); Daniela PISON, Chur (CH)

(73) Assignee: 4DVets AG, Frenkendorf (CH)

(21) Appl. No.: 17/026,568

(22) Filed: Sep. 21, 2020

(30) Foreign Application Priority Data
Sep. 30, 2019 (EP) 19 200 556.9

(51) Int. Cl. A61B 5/11; A61B 5/00

(52) U.S. Cl. CPC A61B 5/1127; A61B 5/112; A61B 2562/0219; A61B 2503/40; A61B 5/6802

(57) ABSTRACT

A device for determining a parameter of the locomotion of animals has at least one marker with a tracker, and/or at least one sensor and an evaluation unit. The marker and/or the sensor are attached to a jacket provided for dressing the animal. The marker and/or sensor is arranged on the jacket so that the marker is arranged on the animal wearing the jacket having a defined spatial assignment to a parameter-relevant body location of the animal associated to the marker and/or sensor. The evaluation unit determines a parameter of the locomotion on the basis of a motion, detected by the tracker, of the parameter-relevant body location associated to the marker and/or on the basis of a motion, detected by the sensor, of the parameter-relevant body location associated to the sensor.

### CROSS-REFERENCE TO RELATED APPLICATIONS

This application claims priority under 35 USC 119 of European Application No. EP 19 200 556.9, filed on Sep. 30, 2019, the disclosure of which is herein incorporated by reference.

### BACKGROUND OF THE INVENTION

The invention relates to a device for determining a parameter of the locomotion of animals, namely, of four-legged mammals, particularly dogs, horses and/or camels,
having at least one marker and a tracker provided for the marker, wherein the marker can be connected to the body of the animal having a defined spatial assignment to a parameter-relevant body location of the animal associated to the marker and by means of the marker a motion, induced by the locomotion of the animal, of the parameter-relevant body location associated to the marker can be detected by the tracker, wherein the motion, induced by the locomotion of the animal, of the parameter-relevant body location associated to the marker is specific to the parameter of the locomotion, and/or
having at least one sensor, which can be connected to the body of the animal having a defined spatial assignment to a parameter-relevant body location of the animal associated to the sensor and by means of which a motion, induced by the locomotion of the animal, of the parameter-relevant body location associated to the sensor can be detected, wherein the motion, induced by the locomotion of the animal, of the parameter-relevant body location associated to the sensor is specific to the parameter of the locomotion, and
having an evaluation unit by means of which the parameter of the locomotion can be determined on the basis of the motion, detected by the tracker, of the parameter-relevant body location associated to the marker and/or on the basis of the motion, detected by the sensor, of the parameter-relevant body location associated to the sensor.

The invention further relates to a jacket for dressing an animal, namely, a four-legged mammal, particularly a dog, a horse and/or a camel, having at least a torso part and a leg part, wherein the torso part is designed to receive the torso of the animal and the leg part is designed to receive a limb of the animal.

For instance, dogs, horses and camels can be impaired in their locomotion due to neurological and orthopedic disorders. Frequent orthopedic disorders of dogs in addition to back disorders are hip and elbow dysplasia, osteochondrosis in the shoulder joint as well as arthrotic and other pathological changes in the hip and knee joints.

It is common practice to analyze the gait of the respective animal in order to examine the health status of the locomotor system and, if applicable, to diagnose orthopedic disorders of animals. In the scope of a gait analysis, parameters of the locomotion of the animal are determined, which are significant in relation to the presence and, if applicable, the severity of an orthopedic disorder. For dogs, for example, the relevant gait parameters are inter alia the step length, the back motion, the time the paws are in contact with the ground, and the length and duration of the step cycle, including the duration of the standing phase of the step cycle and including the length and the joint angle (“range of motion”) and the duration of the swing phase of the step cycle.

A generic device, in the case of which a gait analysis of dogs is performed by means of retroreflectors provided as optical markers and a tracker comprising a plurality of infrared cameras, is described in the article entitled “Pelvic limb kinematics in the dog with and without a stifle orthosis,” published in Veterinary Surgery 2017 vol. 64, pages 642 to 652. Retroreflective markers are attached to the skin of the dog in the range of the right hind leg of the dog concerned, by means of double-sided adhesive tape and cyanoacrylate glue. Further retroreflective markers are attached to an orthosis which is temporarily placed on the right hind leg of the dog. Both the markers attached to the skin of the dog and the markers attached to the orthosis are in a defined position relative to the joints on the leg of the dog. In step and trot, the dog moves along a course, first without and then with the orthosis in place. Thereby the retroreflective markers are detected by means of the infrared cameras. The influence of the orthosis on the gait pattern of the dog, specifically on the motion of the joints on the right hind leg of the dog, is determined by computer from the detected motions of the retroreflective markers.

A generic device that uses inertial sensors to perform gait analyses of dogs is known from the paper entitled “Evaluation of an open source method for calculating physical activity in dogs from harness and collar based sensors,” published in BMC Veterinary Research (2017) 13:322. In this case, motions of dogs are detected by means of four inertial sensors, two of which are attached to a collar put on the dog and two to straps of a harness put on the dog.

Starting from the generic prior art, the object of the present invention is to improve the reproducibility of the detection of parameters of the locomotion of animals, particularly the reproducibility of the detection of gait parameters of dogs, horses and camels.

### SUMMARY OF THE INVENTION

According to the invention, this object is achieved by means of a device for determining a parameter of the locomotion of animals, namely, of four-legged mammals, particularly dogs, horses and/or camels, having at least one marker used for performing a locomotion analysis, particularly a gait analysis, and/or at least one sensor configured to perform a gait analysis, wherein the at least one marker and/or the at least one sensor is attached to a jacket which is put on the animal to be examined. At least one leg, preferably all of the legs of the animal are accommodated by a respective leg part of the jacket, and a torso part of the jacket connected to the leg part(s) is provided for the torso of the animal. The markers and/or the sensors are firmly or detachably connected to the jacket at defined attachment points. The markers are particularly conventional optical markers such as retroreflective markers or color markers, which are detected by means of cameras provided as trackers and which are fixed to the jacket in such a manner that they are accessible to the cameras. Particularly inertial sensors of common design, preferably IMUs that have been proven in practice, can be used as sensors. In a preferred configuration of the invention, the power stores of sensors are rechargeable when the sensor is attached to the jacket. The evaluation of the detected motions is usually computer-aided, with the aid of software commonly used for analyses of this type.

The fit of the jacket is adapted to the body of the animal in such a manner that the aforementioned parts of the jacket interact to fix the jacket positively to the animal wearing the jacket. The form fit ensures that the jacket assumes a defined position on the animal and is secured against undesired slipping. In a preferred configuration of the invention, the fit of at least one of the parts of the jacket is additionally selected in such a manner that the jacket is put on the associated body part of the animal with a preferably low pretension. The material of the jacket can be of an appropriate quality. Jackets or parts of jackets made of stretch fabric which fit snugly to the body of the animal are particularly conceivable, thus ensuring that the jacket is positioned in a defined manner and fixed in place on the animal wearing the jacket. Washable materials are preferred for the jacket.

The defined and fixed in place arrangement of the jacket on the animal wearing the jacket is accompanied by a corresponding arrangement of the at least one marker attached to the jacket and/or the at least one sensor attached to the jacket. Thereby, the attachment locations of the markers and sensors on the jacket are selected in such a manner that the markers and sensors are positioned on the animal wearing the jacket having a defined spatial assignment to parameter-relevant body locations or body parts, for example having a defined spatial assignment to parts of the limbs and to joints of the animal.

The jacket according to the invention ensures a good reproducibility of the determination of locomotion parameters, particularly of gait parameters, in that it is always positioned in the same manner on one and the same individual during chronologically separated locomotion or gait analyses, provided that the constitution of the examined individual has not changed significantly in the meantime. Under this premise, a uniform spatial assignment of the marker(s) attached to the jacket and/or the sensor(s) attached to the jacket to the respective parameter-relevant body locations of the animal to be examined results for all the analyses performed at different times. In the same manner, the jacket can be used to obtain comparable examination results on different individuals having a substantially uniform physique.

The owner of an adult dog, for example, can keep a jacket for this dog that is tailored to the physique of the dog, which jacket provides comparable examination results when the examinations of the animal are staggered over time and thus makes it possible, for example, to gain information about the temporal course of an orthopedic disorder of the animal. Particularly for veterinarians and/or for veterinary research, the invention opens up the possibility of obtaining comparable examination results using one and the same jacket for locomotion or gait analyses performed at different times and/or for different individuals having the same physique.

In one embodiment, the jacket according to the invention has a neck part which, on the animal wearing the jacket, surrounds the neck of the animal and is designed integrally with the leg part(s) and the torso part of the jacket and thereby contributes to the fixed in place arrangement of the jacket on the animal wearing the jacket.

In a further embodiment of the invention, a jacket is provided on which both markers for detection by a tracker and sensors are attached. In this case, it is possible by means of the marker(s) and the associated tracker(s), on the one hand, and by means of the sensor(s), on the other hand, to determine one and the same parameter of locomotion of the animal examined and to use the results obtained separately in this manner for mutual verification thereof.

In a further embodiment of the invention, inertial sensors of common design are preferred as sensors. In the case of one embodiment of the invention, optical, particularly retroreflective markers are used as markers in connection with a camera arrangement provided as a tracker.

A detection of the motion of an optical marker which can be used for analysis purposes can, if the recording arrangement is designed accordingly, for example if simple color markers are used as optical markers, presuppose that the marker is detected by the associated camera arrangement at a certain angle and that the marker and the camera arrangement accordingly have a mutual nominal positioning. In this context, due to a lateral shielding of the marker(s), detection of the motion of the marker(s) by the camera arrangement is only possible if the mutual position of the marker(s) on the one hand and the camera arrangement provided as tracker on the other hand is suitable for obtaining a detection result useful for the motion analysis.

For that purpose, the jacket has, in a further embodiment of the invention, a profile on an outer side thereof which comprises a profile base provided with the optical marker and a profile side wall, which forms the lateral shielding open on the camera side, for the optical marker by, on the animal wearing the jacket, the profile base facing the animal and the profile side wall projecting from the profile base towards the camera arrangement, and by the profile side wall shielding the optical marker from the camera arrangement in the event of a mutual incorrect positioning of the optical marker and the camera arrangement. Preferably, the profile has a U-shaped cross-section and legs of the U-shaped cross-section form the profile side walls and a transverse bar of the U-shaped cross-section connecting the legs forms the profile base.

The functionality of the motion recording device according to the invention may require a special alignment of the marker(s) and/or the sensor(s) on the jacket. For such cases, the jacket and/or the marker(s) or the sensor(s) has (have) adjusting means for the nominal alignment of the marker(s) and/or the sensor(s).

In one embodiment, the sensors are protected in robust receiving pockets. Additionally or alternatively, the pockets can be used to attach the sensors to the jacket with a nominal alignment. Particularly conceivable are an outer contour of a sensor and an inner contour of a receiving pocket, which are matched to each other in such a manner that a sensor inserted into the receiving pocket is inevitably arranged with the nominal alignment thereof on the jacket.

The device according to the invention may comprise a plurality of jackets of different fits and/or different sizes. The device designed in this manner makes it possible to analyze the locomotion of individuals with different physiques. The marker(s) and/or the sensor(s) are preferably detachably attached to the individual jackets. As a result, the same marker(s) and/or the same sensor(s) can be used on all jackets. Apart from the fit and/or size, the jackets of a jacket set can have a uniform configuration.

### BRIEF DESCRIPTION OF THE DRAWINGS

In the following, the invention is explained in more detail using exemplary schematic representations, in which:

FIG. 1 shows a device for performing a gait analysis of dogs comprising a jacket of a first type put on a dog and provided with optical markers and sensors,

FIG. 2 shows a jacket of a second type provided with optical markers and sensors for performing a gait analysis of dogs,

FIG. 3 shows a device for performing a gait analysis of dogs comprising a jacket put on a dog and provided exclusively with sensors,

FIG. 4 shows a jacket provided exclusively with optical markers for performing a gait analysis of dogs, and

FIG. 5 shows a receiving pocket for a sensor which receiving pocket is attached to a jacket as shown in any of FIGS. 1 to 3.

### DETAILED DESCRIPTION OF THE EMBODIMENTS

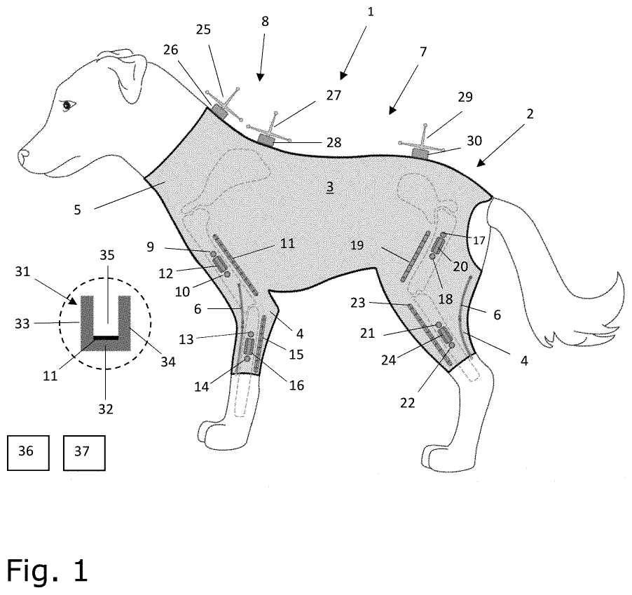

FIG. 1 shows as a device for determining locomotion parameters of four-legged mammals a device 1 for performing gait analysis of dogs.

A substantial part of the device 1 is a jacket 2, which in FIG. 1 is put on a dog to be analyzed regarding the gait thereof.

The jacket 2 is made of textile stretch fabric and has a torso part 3 to accommodate the torso of the dog, a leg part 4 for each of the limbs of the dog and a neck part 5, which encloses the part of the neck of the dog that is close to the torso. To make it easier to put on the jacket 2, each of the leg parts 4 is provided with a zipper 6. In FIG. 1 the zippers 6 are closed. Due to the fit thereof and the elasticity of the material, the jacket 2 fits snugly to the body of the dog, thereby having a low pretension and being secured against undesired slipping.

With respect to fit and/or size, the jacket 2 is ready-made and adapted to a common physique of dogs of a certain weight. Other jackets 2 of the device 1, not shown in FIG. 1, have a different fit or size from the jacket 2 shown and are intended for dogs of higher or lower weight.

At defined attachment points optical markers 7 are attached to the jacket 2 as markers and inertial sensors as sensors 8. The attachment points for the optical markers 7 and the sensors 8 are selected in such a manner that the optical markers 7 and the sensors 8 are attached to the dog wearing the jacket 2 having a defined spatial assignment to parameter-relevant body locations of the dog.

In the present example, the range of motion of the legs of the dog during the swing phase of the step cycle is to be determined as a parameter of the locomotion of the dog by means of a gait analysis. Specific to this parameter are the motions of the upper and lower arms, thighs, shoulders, neck and pelvis of the dog. This results in the parameter-relevant body locations of the dog to be detected in the gait analysis.

The following optical markers 7 and sensors 8 having a defined spatial assignment to the parameter-relevant body locations of the dog are attached to the left side of the dog visible in FIG. 1 by means of the jacket 2:
at the level of the left upper arm bone (humerus): optical humerus markers 9, 10, 11 and a humerus sensor 12;
at the level of the left spoke (radius): optical radius markers 13, 14, 15 and a radius sensor 16;
at the level of the left upper thigh bone (femur): optical femur markers 17, 18, 19 and a femur sensor 20;
at the level of the left shinbone (tibia): optical tibia markers 21, 22, 23 and a tibia sensor 24.

Corresponding optical markers 7 and sensors 8 are attached to the part of the jacket 2 assigned to the right side of the body of the dog having a defined spatial assignment to the parameter-relevant body locations of the dog there.

Additionally, the jacket 2 is provided on the back of the dog with the following sagittal markers 7 and sensors 8, which have a defined spatial assignment to the parameter-relevant body locations of the dog:
towards the neck (cervical): a cervical optical marker 25 and a cervical sensor 26;
in the range of the shoulder blades (scapular): a scapular optical marker 27 and a scapular sensor 28;
in the range of the sacrum (sacral): a sacral optical marker 29 and a sacral sensor 30.

The optical humerus markers 9, 10, the optical radius markers 13, 14, the optical femur markers 17, 18, the optical tibia markers 21, 22, the cervical optical marker 25, the scapular optical marker 27 and the sacral optical marker 29 are spherical retroreflective markers of conventional design. As optical humerus marker 11 and also as optical radius marker 15, as optical femur marker 19 and as optical tibia marker 23, in each case a reflective red colored stripe is provided on the jacket 2.

Representing the last aforementioned four markers, the dotted framed partial representation of FIG. 1 shows the optical humerus marker 11. Accordingly, the humerus marker 11 is embedded in a profile 31 made of plastics, which has a U-shaped cross-section. The humerus marker 11 is affixed to a profile base 32 formed by a transverse bar of the U-shaped cross-section. At the jacket 2 put on the dog, a profile opening 35 bordered by profile side walls 33, 34 points to a camera arrangement 36 which is shown quite schematically in FIG. 1. In a corresponding manner, the optical radius marker 15, the optical femur marker 19 and the optical tibia marker 23 are attached to a profile 31 attached to the jacket 2.

The camera arrangement 36 is provided as a tracker for detecting the motion of the optical markers 7 induced by a motion of the dog and therefore for detecting the motion of the parameter-relevant body locations of the dog associated to the optical markers 7. In a common manner, the camera arrangement 36 comprises a plurality of cameras placed along a course to be passed by the dog during the gait analysis.

The camera arrangement 36 detects the motion of all optical markers 7. The detection of the optical humerus marker 11, the optical radius marker 15, the optical femur marker 19 and the optical tibia marker 23 by the camera arrangement 36, however, only provides a usable result for gait analysis under the condition that the camera arrangement 36 views the respective optical marker 7 at a nominal angle.

Profiles 31 ensure that the detection of the optical humerus marker 11, the optical radius marker 15, the optical femur marker 19 and the optical tibia marker 23 by the camera arrangement 36 is performed at the nominal angle. Only if the camera arrangement 36 is aligned with the optical markers in question at the nominal angle, the optical humerus marker 11, the optical radius marker 15, the optical femur marker 19 and the optical tibia marker 23 are accessible for detection by the camera arrangement 36 through the respective profile opening 35. Otherwise, said markers are shielded from the camera arrangement 36 by one of the profile side walls 33, 34 of the profile 31.

The sensors 8 attached to the jacket 2 are conventional inertial measurement units (IMUs) each having three translation sensors and three gyroscopic sensors. Also conceivable are magnetic sensors designed as 9d sensors, for example.

For determining the gait parameter at issue, the motions of the parameter-relevant body locations of the dog, detected by means of the camera arrangement 36 and the associated optical markers 7, and the motions of the parameter-relevant body locations of the dog, detected by means of the sensors 8, are evaluated separately from one another in a numerical evaluation unit 37 computer-aided with the aid of conventional software. The two separately determined results of the parameter determination are used for mutual verification.

To ensure appropriate alignment of the optical markers 7 and the sensors 8 on the jacket 2, markings not shown are provided on the jacket 2 along which markings the optical markers 7 and the sensors 8 are to be aligned.

In order to protect, in particular, the sensors 8 against harmful external influences, it is conceivable that corresponding receiving pockets are provided on the jacket 2, which can additionally ensure a nominal alignment of the sensors 8.

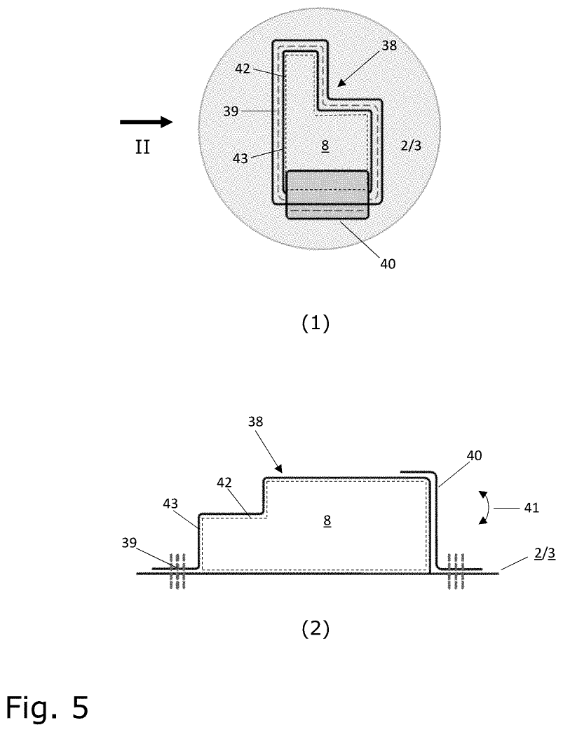

FIG. 5 shows an example of such a receiving pocket 38 having a sensor 8 accommodated therein. Partial representation (1) of FIG. 5 shows the receiving pocket 38 and the sensor 8 in a top view of the jacket 2 provided with the receiving pocket 38. In the partial representation (2) of FIG. 5, the receiving pocket 38 including the sensor 8 is shown in a view in the direction of the arrow II in the partial representation (1).

Along a seam 39 the receiving pocket 38 is attached to the torso part 3 of the jacket 2. A cover flap 40 of the receiving pocket 38 can be pivoted in the direction of a double arrow 41 to an open position or to the closed position shown in FIG. 5. In the closed position the cover flap 40 is fixed to the remaining receiving pocket 38 by means of a hook and loop fastener. The nominal alignment of the sensor 8 is achieved by the interaction of an outer contour 42 of the sensor 8 and an inner contour 43 of the receiving pocket 38 matched to the outer contour 42 of the sensor 8. The outer contour 42 of the sensor 8 and the inner contour 43 of the receiving pocket 38 form adjusting means and, as such, ensure that the sensor 8 can only be inserted into the receiving pocket 38 with an alignment relative thereto due to which the sensor 8 achieves its nominal alignment when inserted.

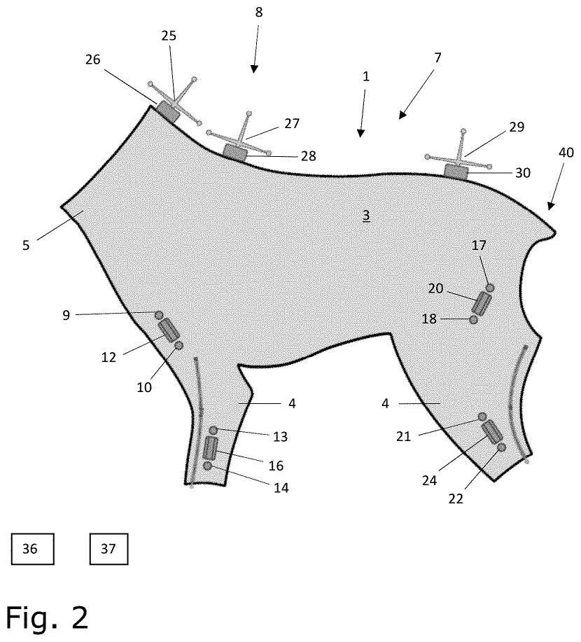

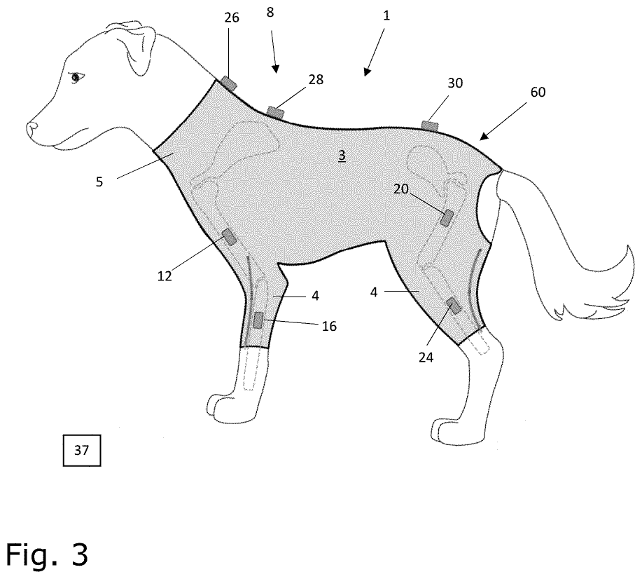

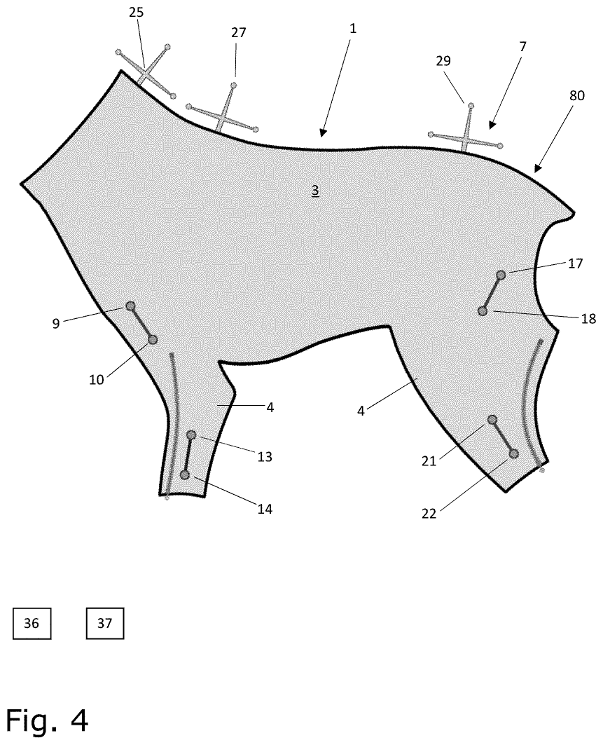

Instead of the jacket 2 described in detail above, the device 1 for performing a gait analysis of dogs may comprise a jacket 40 as shown in FIG. 2, a jacket 60 as shown in FIG. 3 or a jacket 80 as shown in FIG. 4.

The jacket 40 shown in FIG. 2 differs from the jacket 2 shown in FIG. 1 only due to the absence of the optical humerus marker 11, the optical radius marker 15, the optical femur marker 19 and the optical tibia marker 23. For the attachment and nominal alignment of the sensors 8 to the jacket 40, receiving pockets 38 not shown in FIG. 2 can be used as shown in FIG. 5.

When using the jacket 60 according to FIG. 3, the detection of the motion of the parameter-relevant body locations of the dog is performed without any optical markers 7 and therefore exclusively by means of the sensors 8 also provided on the jacket 2 according to FIG. 1, which sensors 8 are also arranged on the jacket 60 hays ing a defined spatial assignment to the parameter-relevant body locations of the dog wearing the jacket 60. Apart from that, also the jacket 60 corresponds to the jacket 2 as shown in FIG. 1 in design and function. Particularly the jacket 60 may also be provided with receiving pockets 38 of the type shown in FIG. 5 for the attachment and nominal alignment of the sensors 8.

Only the spherical retroreflective optical markers 7 shown in FIG. 1 are attached to the jacket 80 shown in FIG. 4, the optical markers 7 being the optical humerus markers 9, 10, the optical radius markers 13, 14, the optical femur markers 17, 18, the optical tibia markers 21, 22, the cervical optical marker 25, the scapular optical marker 27 and the sacral optical marker 29. Accordingly, the device 1 for the gait analysis provided with the jacket 80 does not make use of sensors 8. Rigid connections hidden in FIG. 1 by the humerus sensor 12, the radius sensor 16, the femur sensor 20 and the tibia sensor 24 and provided between the optical humerus markers 9, 10, the optical radius markers 13, 14, the optical femur markers 17, 18 and the optical tibia markers 21, 22 are visible in FIG. 4.

What is claimed is:

1. A device for determining a parameter of the locomotion of animals, namely, of four-legged mammals, particularly dogs, horses and/or camels, comprising:
at least one marker and a tracker provided for the marker, wherein the marker is configured to be connected to a body of the animal having a defined spatial assignment to a parameter-relevant body location of the animal associated to the marker and by means of the marker the tracker is configured to detect a motion, induced by the locomotion of the animal, of the parameter-relevant body location associated to the marker, wherein the motion, induced by the locomotion of the animal, of the parameter-relevant body location associated to the marker is specific to the parameter of the locomotion, and/or
at least one sensor, which is configured to be connected to the body of the animal having a defined spatial assignment to a parameter-relevant body location of the animal associated to the sensor and by means of which a motion, induced by the locomotion of the animal, of the parameter-relevant body location associated to the sensor can be detected, wherein the motion, induced by the locomotion of the animal, of the parameter-relevant body location associated to the sensor is specific to the parameter of the locomotion, and
an evaluation unit by means of which the parameter of the locomotion can be determined on the basis of the motion, detected by the tracker, of the parameter-relevant body location associated to the marker and/or on the basis of the motion, detected by the sensor, of the parameter-relevant body location associated to the sensor, wherein
the marker and/or the sensor are configured to be connected to the body of the animal by attaching the marker and/or the sensor to a jacket provided for dressing the animal,
wherein the jacket comprises at least a torso part and a leg part, wherein the torso part is designed to receive the torso of the animal and the leg part is designed to receive a limb of the animal, and
wherein the marker is arranged on the jacket so that the marker is arranged on the animal wearing the jacket having the defined spatial assignment to the parameter-relevant body location of the animal associated to the marker and/or wherein the sensor is arranged on the jacket so that the sensor is arranged on the animal wearing the jacket having the defined spatial assignment to the parameter-relevant body location of the animal associated to the sensor.

2. The device according to claim 1, wherein the jacket has a neck part for at least partially receiving a neck of the animal.

3. The device according to claim 1, wherein at least one said marker and at least one said sensor are provided, and wherein the marker and the sensor are arranged on the jacket so that, on the animal wearing the jacket, the marker is arranged having the defined spatial assignment to the parameter-relevant body location of the animal associated to the marker and the sensor is arranged having the defined spatial assignment to the parameter-relevant body location of the animal associated to the sensor,
wherein the motion, induced by the locomotion of the animal, of the parameter-relevant body location associated to the marker and the motion, induced by the locomotion of the animal, of the parameter-relevant body location assigned to the sensor are specific to one and the same parameter of the locomotion, and
wherein the evaluation unit is configured to determine this parameter of the locomotion both on the basis of the motion, detected by the tracker, of the parameter-relevant body location associated to the marker and on the basis of the motion, detected by the sensor, of the parameter-relevant body location associated to the sensor.

4. The device according to claim 1, wherein at least one of the at least one sensor is designed as an inertial sensor.

5. The device according to claim 1, wherein at least one of the at least one marker is designed as an optical marker and that a camera arrangement is provided as the tracker.

6. The device according to claim 5, wherein the optical marker has a lateral shielding open on a camera side, which, in the event of a mutual nominal positioning of the optical marker and the camera arrangement, permits detection of the optical marker by the camera arrangement and which, in the event of a mutual incorrect positioning of the optical marker and the camera arrangement deviating from the mutual nominal positioning, shields the optical marker from the camera arrangement.

7. The device according to claim 6, wherein the jacket has a profile on an outer side thereof which comprises a profile base provided with the optical marker and a profile side wall, which forms the lateral shielding open on the camera side, for the optical marker by, on the animal wearing the jacket, the profile base facing the animal and the profile side wall projecting from the profile base towards the camera arrangement, and by the profile side wall shielding the optical marker from the camera arrangement in the event of a mutual incorrect positioning of the optical marker and the camera arrangement.

8. The device according to claim 7, wherein the profile has a U-shaped cross-section and that legs of the U-shaped cross-section form the profile side walls and a transverse bar of the U-shaped cross-section connecting the legs forms the profile base.

9. The device according to claim 1, wherein adjusting means are provided on the jacket and/or on the marker or the sensor, by means of which a nominal alignment of the marker attached to the jacket and/or of the sensor attached to the jacket can be defined.

10. The device according to claim 1, wherein the jacket is provided with a receiving pocket for the sensor.

11. The device according to claim 1, wherein a selection of a plurality of jackets is provided which have a different fit and/or which are adapted to different body sizes and/or weights of animals to be dressed.

12. A jacket for dressing an animal, namely, a four-legged mammal, particularly a dog, a horse and/or a camel, having at least a torso part and a leg part, wherein the torso part is designed to receive the torso of the animal and the leg part is designed to receive a limb of the animal, wherein at least one marker of a device for determining a parameter of the locomotion of the animal is attached to the jacket so that the marker is arranged on the animal wearing the jacket having a defined spatial assignment to a parameter-relevant body location of the animal associated to the marker and, by means of the marker, a tracker is configured to detect a motion, induced by the locomotion of the animal, of the parameter-relevant body location associated to the marker, wherein the motion, induced by the locomotion of the animal, of the parameter-relevant body location associated to the marker is specific to the parameter of the locomotion of the animal and/or wherein at least one sensor of a device for determining a parameter of the locomotion of the animal is attached to the jacket so that the sensor is arranged on the animal wearing the jacket having a defined spatial assignment to a parameter-relevant body location of the animal associated to the sensor and, by means of the sensor, a motion, induced by the locomotion of the animal, of the parameter-relevant body location associated to the sensor can be detected, wherein the motion, induced by the locomotion of the animal, of the parameter-relevant body location associated to the sensor is specific to the parameter of the locomotion of the animal.
